(19) United States
(12) Patent Application Publication
(10) Pub. No.: US 2006/0085047 A1
(43) Pub. Date: Apr. 20, 2006

(54) Neuromuscular electrical stimulation of the foot muscles for prevention of deep vein thrombosis and pulmonary embolism with motion detection control

(76) Inventors: John D. Unsworth, Hamilton (CA); Robert Edward Kaplan, Buffalo, NY (US); James Joseph Czyrny, Amherst, NY (US); Jack Hirsh, Burlington (CA)

Correspondence Address: JOHN D. UNSWORTH, LL.B.;Suite 107, 7 Innovation Drive, Dundas, ON, L9H 7H9, CA

(21) Appl. No.: 10/966,143

(22) Filed: Oct. 18, 2004

(51) Int. Cl. A61N 1/08

(52) U.S. Cl. 607/48; 607/144

(57) ABSTRACT

The invention describes a method to automatically controlling, single channel Neuromuscular Electrical Stimulation (NMES) of the plantar muscle, in response to the sensing of motion of the foot or leg: to reduce accommodation of the stimulated plantar muscle and attendant reduction of contractions, which when undiminished increase blood flow for the prevention of Deep Vein Thrombosis (DVT) and Pulmonary Embolism (PE); to turn off the stimulation during walking or running to prevent slips or falls; and to reduce power consumption of the unit that provides the stimulation.

### FIELD OF THE INVENTION

This invention relates to the prevention of Deep Vein Thrombosis (DVT) and Pulmonary Embolism (PE) by increasing blood flow in the lower leg.

### BACKGROUND OF THE INVENTION

This invention relates to a method of automatically controlling the delivery of, single channel Neuromuscular Electrical Stimulation (NMES) of the plantar muscle, in response to the sensing of motion of the foot or leg: to reduce accommodation of the stimulated plantar muscle and attendant reduction of contractions, which when undiminished increase blood flow for the prevention of Deep Vein Thrombosis (DVT) and Pulmonary Embolism (PE); and to turn off the stimulation during walking or running to prevent slips or falls and to reduce power consumption of the unit that provides the stimulation.

Venous thromboembolic disease (VTED) continues to be a cause of significant morbidity and mortality for individuals immobilized during prolonged travel, after orthopedic surgery, neurologic disorders, and a variety of other conditions.

U.S. Pat. No. 6,615,080 describes a method of reducing the incidence of Deep Vein Thrombosis (DVT) and Pulmonary Embolism (PE) by the application of electrical stimulation routine of the plantar muscle. While the method described in the said patent is effective, if the plantar muscle is stimulated for an excessive amount of time the muscles accommodate to the stimulation and become less responsive to the application of electrical stimulation.

There is also the issue of safety; for example, the user's coordination while walking may be affected by the muscular contractions that occur as a result of electrical stimulation of the plantar muscle, however slight. Such interference could cause a slip or a fall.

In addition, applying the electrical stimulation routine when the foot and or leg are in substantial motion, and the stimulation is not required, wastes battery life and imposes constrains on the use of the method when long periods of treatment are required and direct connection to a power source is inconvenient.

What is needed therefore is a method of applying an electrical stimulation routine to the plantar muscle, as described in U.S. Pat. No. 6,615,080, or for other methods of electrically simulating the muscles of the foot, only when the foot and or lower leg are substantially stationary and turning off the electrical stimulation, when the foot and/or lower leg is substantially in motion.

Therefore, automatically turning the electrical stimulator on and off, in response to motion detection, will: ensure that the electrically stimulated muscles are less subject to the accommodation effect attendant with prolonged electrical stimulation of muscles; ensure a safer treatment as the electrical stimulation will turn off, when the user is walking or running; ensure that it will be better tolerated by the user, as electrical stimulation will only be administered, when it is beneficial; and ensure that power consumption is reduced by being on only when required, which will mean that battery powered units will have a far greater effective usage time, without replacement of batteries or recharging.

The U.S. Pat. No. 6,615,080 describes a method for preventing DVT, PE, ankle edema and venostasis and a device that includes a single channel sequential neuromuscular electrical stimulation (NMES) unit. The NMES unit 10 can be any NMES unit that is battery powered, compact and can be programmed to deliver the stimulus profile described below or such other profile that is found to be efficacious, such as the Focus™ manufactured by Empi Inc., 599 Cardigan Road St. Paul, Minn., U.S.A. In order to simplify the patient's ability to properly apply the NMES device, the stimulator generates biphasic symmetrical square wave pulses with stimulus parameters that our study demonstrated to result in optimum venous blood flow. The stimulus frequency is fixed at 50 pulses per second, the stimulus duration is set at 300 microseconds, the ramp up time at 2 seconds, the ramp down time at 2 seconds, and the stimulus cycle set at 12 seconds on and 48 seconds off. Once set in advance by the Doctor, manufacturer or user, the only adjustment necessary on the part of the patient is a stimulus intensity dial. This allows for a current up to 20 milliamperes to be delivered. The user adjusts the intensity to the point needed to produce a minimally visible or palpable muscle contraction. The output leads of the stimulator are attached through a conductor to electrodes of various types including, self-adherent surface electrodes. These electrodes being of opposite polarity and creating an electrical potential difference between themselves and the tissue that separates them. The frequency and electrical characteristics of electrical impulses applied to the patient is herein referred to as the electrical stimulation routine.

While the type of electrical pulse generating unit and those characteristics and routine for administering the pulses described above have been found to be very effective in increasing blood flow, it is to be understood that any pulse generator that causes the foot muscle to periodically and gently contract, such that the user does not experience excessive pain, and that includes a motion detection and control means that interrupts the routine when the foot and or lower leg are in motion, is within the ambit of the invention herein disclosed.

### BRIEF DESCRIPTION OF THE INVENTION

The present invention is a method of automatically applying the electrical stimulation routine of the plantar muscle, described in U.S. Pat. No. 6,615,080 or for similar methods of electrically stimulating the muscles of the foot for the prevention and treatment of DVT, PE, ankle edema and venostasis (hereinafter for convenience referred to collectively as “DVT”), which patent is incorporated herein by specific reference. Since it is the inactivity of the calf muscles that cause DVT, any means to turn on and off the electrical stimulation, must detect either muscle activity in the calf muscle, or gross motion of the lower leg, foot or both. Since the foot and lower leg are attached, for practical purposes the motion detection means can be attached to either or both, since a moving foot will be attendant with movement in the calf muscle. The preferred embodiment is to include the motion detection means into the neuromuscular electrical stimulation (NMES) unit 10, which can be attached to the foot or leg, by cuffs or as part of a sock or other body covering, such as a shoe, boot or cast. For the purposes of this disclosure, it is assumed that the motion detection means is incorporated into the NMES device, but it is to be understood that some embodiments of the invention include motion detection means that are separate, but in communication with the NMES device by wire, wirelessly, or by other means well known to the art.

One preferred embodiment of the invention incorporates a solid state motion detection sensor or accelerometer that is incorporated into or connected to the NMES unit. This NMES unit turns the NMES unit off automatically (after being manually turned on by the user), and interrupting and/or delaying the preprogrammed electrical stimulation routine, when motion is detected of such duration, frequency, amplitude or force, or combination or subset thereof, that exceeds a predetermined threshold or map of thresholds. Motion detectors or accelerometers and control circuits of the type required for the preferred embodiment are all well known to the art and include micro electromechanical systems (MEMS) that are digital or analogue. Motion detectors or accelerometers suitable for this purpose might have single or multiple axis detection, depending upon the use to which it is designed to be used. The MEMS devices, referred to, are extremely compact and inexpensive and can be readily integrated with a processor, controller 10b to turn on and off the NMES unit 10, which processor or controller, including associated memory element(s) 10c, may itself contain the electrical stimulation routine instructions. For example, the unit might have a preprogrammed map that would interrupt the electrical stimulation routine for a combination of motions that would connote walking, but would not interrupt the routine when a foot is simply fidgeting. The ideal map and associated algorithms that compare the map with actual sensory inputs, and direct electrical stimulation events, can be varied to accommodate particular types of uses and patients with particular needs. This map and algorithms could be fixed in the memory element(s) associated with the processor or controller or it could be subject to reprogramming and adjustment by an operator, while in use, using means well known to the art, including infrared remote controls. This would be particularly helpful for patients that suffer from neuromuscular conditions, such as Parkinson's disease. Other preferred embodiments use other types of motion detectors or combination of them, for example, strain gauge sensors and/or pressure sensors, that either directly or indirectly sense motion.

### BRIEF DESCRIPTION OF THE DRAWINGS

FIG. 1 is a perspective view that illustrates a typical location of the electrodes on the sole of the foot 6a, 6b, that being on the area over or proximal to the intrinsic muscles on the plantar surface of the foot.

FIG. 2 is a perspective view that illustrates the conventional self-adhering electrode 6 with conductor 7.

FIG. 3 is a perspective view that illustrates a preferred embodiment of the invention that locates the NMES device 10 in a pocket or pouch in an item of footwear, which incorporated a processor or controller 10b, and with conductors connecting the said NMES device to two electrodes placed beneath the heel and the area around the ball of the foot.

FIG. 4 is a perspective view that illustrates a preferred embodiment of the invention that includes a strain sensor 10g type of motion detector.

FIG. 5 is a perspective view that illustrates a preferred embodiment of the invention that locates the NMES device 10 on the top of the foot, and attaches it to an item of footwear with a complementary hook and loop material 14a, 14b, such as VELCRO. FIG. 5 includes a pressure-motion sensor 10f.

FIG. 6 is a diagrammatical representation of the electronic components: the NMES unit 10, in which controller 10b automatically turns off and on the said unit (after being initially turned on by the user) in response to motion, detected by motion detectors 10a, 10f or 10g or a subset of them or other motion detectors, in cooperation with the electrical stimulation routine.

### DETAILED DESCRIPTION OF THE PATENT

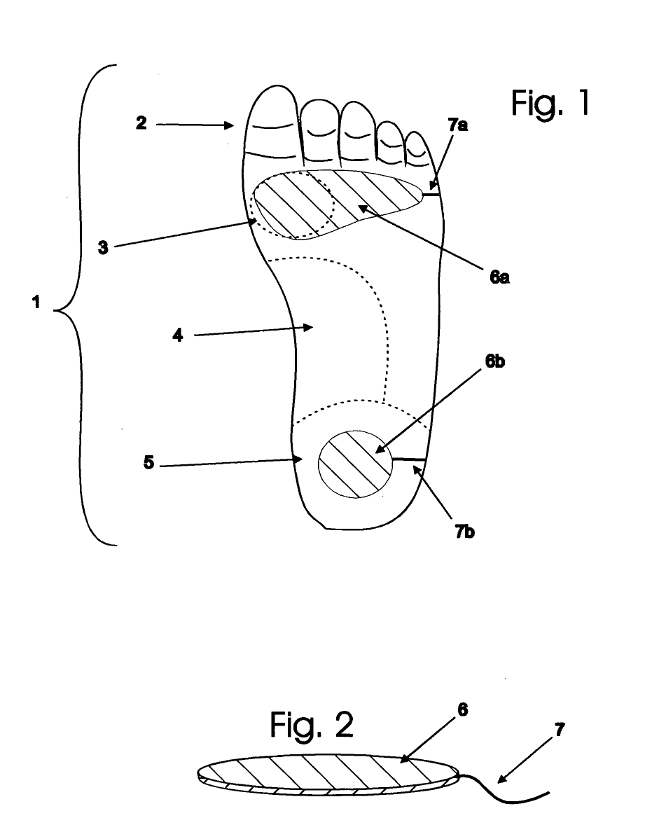

FIG. 1 illustrates the areas of the bottom of the foot where the electrodes 6a and 6b are placed that deliver the electrical impulses generated by the NMES device 10. It is to be understood that these are approximate locations and sizes of electrodes and preferred embodiments of the invention will be of different sizes and shapes, and still come within the ambit of the invention. In some preferred embodiments of the invention the electrode 6a occupies only the area of the ball of the foot, while other preferred embodiments are elliptical in shape, having their major axis normal to the longitudinal axis of the foot 1. It is to be understood that the electrodes may be located in any configuration provided that they stimulate the plantar muscle, and any such configuration would come within the ambit of the invention.

In preferred embodiments of the system and devices that are described herein and that effect the method which, together with those devices form the subject matter of this invention; have electrodes placed on a “platform” with which the user's foot is in substantial contact, which includes contact separated by a garment or material such as a sock or stocking. This platform can be the sole of a shoe, slipper, sock, stocking, cast, pressure or compression stocking, any other item of footwear or it can be the part of an item that is inserted into any such item of footwear that contains electrodes, as described herein, or be the electrodes themselves. It is to be understood then that the planar surface on which the electrodes are attached or detachably attached and that come into contact with the bottom of the user's foot directly or by some part of the electrode is a “platform” for the purposes of this patent.

The electrodes in some preferred embodiments of the invention are the standard self-adhering, and somewhat sticky electrodes that are generally used for such purposes, as illustrated in FIG. 2 The electrodes 6a and 6b can be fabricated from a conducting foil and a conducting hydrogel adhesive, or from any other suitable conducting medium or could be on of myriad conventional electrodes that could be utilized for transcutaneous electro-neural stimulator units (TENS) devices such as those produced by the 3M company. Each of the illustrations contains features of preferred embodiments, but it is to be understood that some of these features could be incorporated into the other preferred embodiments and features deleted from those preferred embodiments or both. While the illustrations show one foot it is to be understood that for most applications each foot will have its own NMES unit 10 with connected motion sensor 10a and controller 10b.

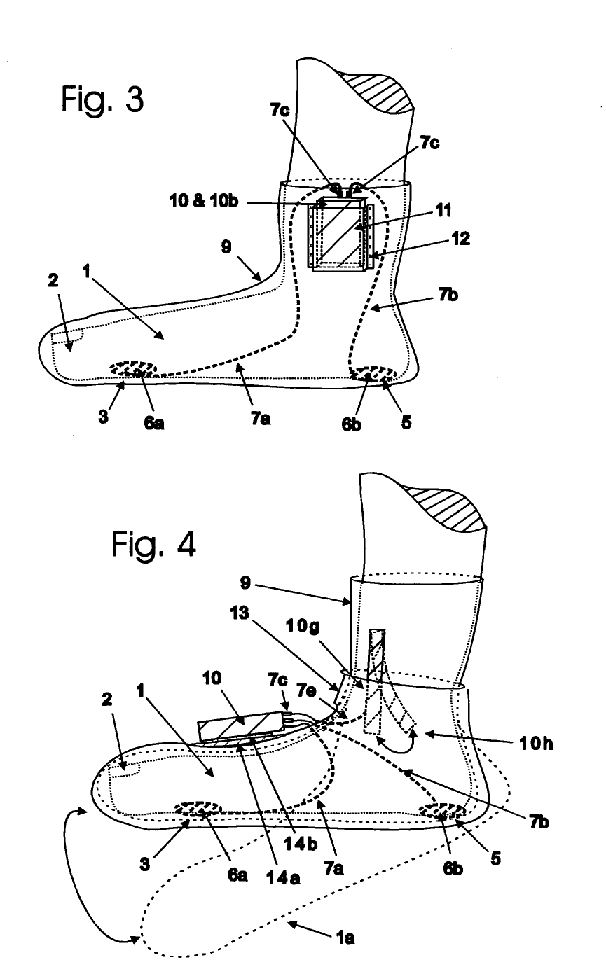

FIG. 3 to FIG. 5 are only meant to be illustrative of the many ways in which the platform containing the electrodes or the electrodes can be applied to the foot and possible locations of sensors on the foot and foot covering. Preferred embodiment of the invention includes and combination of the elements shown on each of the illustrations.

FIG. 3 illustrates an item of footwear which could for example be a sock, slipper or shoe. The item of footwear could extend any convenient length up the ankle or leg. The conductors 7a and 7b can be partly or completely incorporated into the material from which the footwear is fabricated, attached to it or be completely or partly separate from it. The said conductors 7a and 7b connect the electrodes 6a and 6b to the NMES unit 10. The connection of the conductors 7a and 7b at the NMES unit 10 is usually by means of a standard plug connector 7c. The contacts 6a and 6b for this preferred embodiment would be the standard disposable self-adhering electrodes. These electrodes 6a, 6b could be separate from the said item of footwear 9 or it could be incorporated into it, in which case the footwear would likely be disposable. The said electrodes 6a and 6b would be placed in a similar manner as the preferred embodiment of the invention illustrated in FIG. 1. The NMES unit 10 in this preferred embodiment is placed in a pocket or pouch 11 that is attached to by standard fastening means 10 or incorporated into the material of the footwear 9. But another preferred embodiment might detachably attach the said NMES unit 10 to the footwear 9 using a patch of hook or loop material, attached to the said NMES unit 10, and a complementary patch of loop or hook material being attached to the said footwear 9, or forming part of the said footwear. In preferred embodiments of this invention, including the preferred embodiment illustrated in FIG. 3, the NMES unit 10, is connected to a motion detector, an accelerometer 10a or strain sensor 10g (as illustrated in FIG. 4) and controller 10b, that processes the motion detector's signals, and in accordance with a programmed algorithm turns off and on the NMES unit using a programmed map of sensor signals that connote sufficient or insufficient motion of the foot or lower leg in particular directions, or by similar means, well known to the art. The controller 10b turns off the unit when natural blood circulation in the lower leg is presumed, by the nature of the detected motion, to be sufficient to prevent DVT and perhaps, for safety, when the sensor signals, when compared against the programmed map, indicate that the patient is walking or running. The controller 10b turns on the unit when natural blood circulation in the lower leg is presumed to be insufficient to prevent DVT, and perhaps when the sensor signals, when compared against the map, indicate that the patient is not walking or running. In summary, the control algorithm is programmed to optimize the delivery of electrical stimulation to minimize electrical usage, minimize accommodation, perhaps turn off the stimulation during subject ambulation, and maximize blood flow to prevent DVT, all for the particular subject or class of subjects for whom the treatment and device is designed

It is to be understood that when the controller 10b turns the unit NMES unit 10 on and off, it can either do so in a manner which merely interrupts the electrical simulation routine, not otherwise effecting its timing, or it might postpone the routine, that is, delay the routine by the amount of time the controller has turned on or off the NMES unit 10, or the controller 10b could use some combination of interrupting and postponing of the routine, depending upon the requirements of a particular patient or group of patients. It is to be understood that turning on and off the unit 10, does not for the purposes of this disclosure, mean that in all cases the unit is completely turned on and off, rather that certain functions that are necessary for the delivery of stimulation are, at some point in the process, turned on and off.

In the preferred embodiment of the invention the controller or processor, 10b is connected to or has integrated into it, a memory unit(s) 10c, and timer 10d. The memory element(s) holds the map, operating instructions, and controlling algorithms; and the processor or controller compares the map of putative sensor readings that connote various motions of the foot or calf muscle with the actual censor readings to determine the on off mode for directing the NMES unit 10 to deliver or not deliver stimulation. Some preferred embodiments of the invention include an interface 10e that can be a switch, infrared port, wireless port or other interface or control input, or both, well known to the art. This interface 10e is used to instruct the processor or controller as to its on off condition, load and perhaps reload the map and operating settings, and perhaps communicate with the unit 10 on the other foot to coordinate, in some respects, the functioning of each unit. This interface 10e could also contain a wireless interface to a separate, user control unit, which would permit the user to vary the setting of the unit(s) 10 remotely, without bending down. It should be understood that the controller or processor, the memory unit and timer, as well as the communications interface, and their functions can each be separate components and functions, integrated into each other, integrated within the NMES unit 10, or both.

Some preferred embodiments of the invention have motion detector(s) that are comprised of strain gauge sensor(s) that indicate bodily movement or body bending. These strain sensors are well known to the art and are available in many configurations, including those made from optical fibers, piezoelectric, piezoresistive materials, magnetic-electrical components, and materials that change their electrical properties, such as resistance, capacitance, or their optical properties in response to strain. These can be attached, woven, knitted or integrated into any body covering including a sock or cuff or directly attached to the body. For example, the preferred embodiment illustrated on FIG. 4, includes a strip type strain sensor 10g which can be connected to the processor 10b, in NMES unit 10, by a connector 7e. In the preferred embodiment illustrated on FIG. 4, the strain sensor 10g is attached to the sock 9 on top of the ankle. When the ankle articulates and the foot 1a moves, the strain sensor 10g on FIG. 4, which is attached to the sock adjacent to the ankle, bends 10h as well, thus sending a signal to the processor 10b that part or the entire ankle is bending and therefore the calf muscle is presumed to be moving. While the preferred embodiment illustrated on FIG. 4 has a strain sensor 10g on the ankle, it is to be understood that one or more such strain sensors could be located anywhere on the foot or leg provided that it would indicate movement or activity of the calf muscle. Although FIG. 4 illustrates a strain sensor that is connected to the NMES unit 10 by connector 7e, some embodiments of the invention have strain sensor strips or threads that are directly connected to the said unit. If a strain sensor(s) are utilized to indicate calf muscle activity, the processor 10b compares the signals sent by the strain sensor(s) 10g and compares these to a map of putative preprogrammed sensor inputs contained in memory that would correspond to predetermined levels of body motion, or similar methods well known to the art. The processor after making the comparison directs the NMES unit 10 to deliver or not deliver electric stimulation to the plantar muscle via electrodes 6a and 6b. The controller or processor 10b then instructs the NMES unit 10 not to deliver electrical stimulation of the plantar muscle if the calf muscle is deemed to be in sufficient motion that DVT would not likely occur, or deliver electrical stimulation to the plantar muscle if the calf muscle is deemed to be sufficiently immobile that DVT is sufficiently probable and perhaps if the subject is not walking, running or ambulating. As in the case of the preferred embodiment that contains an accelerometer, described above, the preferred embodiment that employs a strain sensor, can be integrated with other motion detectors, and with the electrical stimulation routine to deliver electrical stimulation of the plantar muscle, only when needed to prevent DVT, and it is safe to do so, that is the subject is not ambulating. The preferred embodiment of the invention could also contain an interface 10e with those features described above.

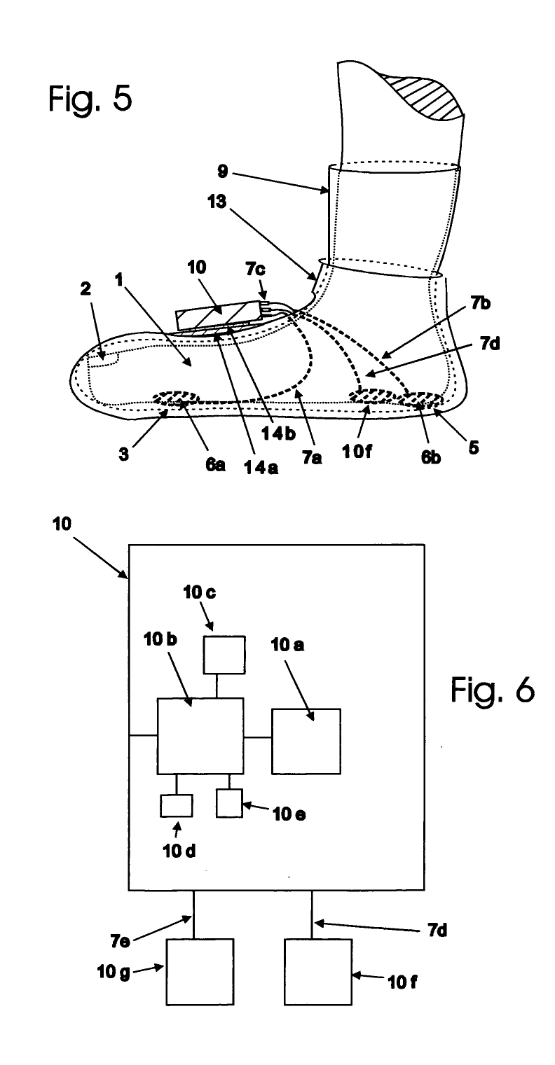

Some preferred embodiments of the invention have a pressure-motion sensor 10f attached to NMES unit 10 by connector 7d, as illustrated in FIG. 5 and FIG. 6 or such connection from the pressure-motion sensor 10f to the NMES unit 10 could be wireless. While only one pressure-motion sensor 10f is shown on FIG. 5, any number of sensors could be utilized, and be placed on different parts of the foot, as could sensor 10f; and the pressure-motion sensor could be combined with one or more of the electrodes 6a, 6b, rather than being separate, as is shown in FIG. 5. This pressure-motion sensor, if incorporated into a preferred embodiment, can supplement the motion sensor 10a and/or 10g or replace one or both of them. This pressure-motion sensor might also be supplement by a Global Positioning Sensor (GPS) or other motion detection technology, known to the art, and integrated into the NMES unit 10.

This pressure-motion sensor 10f can indicate whether the person is putting sufficient weight on the foot to indicate that the user is, for example, standing or walking and the feet and legs are in motion. In most preferred embodiments when the motion-pressure indicator indicates that the pressure exerted on the motion-pressure sensor 10f is consistent with that which would be exerted when the subject is standing on the sensor, the delivery of electrical stimulation would be interrupted immediately, to avoid the danger of a muscle contraction interfering with normal walking or ambulation. In most preferred embodiments, the electrical stimulation is turned on when the motion-pressure indicator 10f indicates that the pressure exerted on the sensor 10f is consistent with that which would be exerted when the subject is not standing on the sensor 10f for a period of time, for example, 20 seconds. This would indicate that the person is not standing, but may be sitting or prone. In most preferred embodiments, if the motion-pressure sensor 10f indicates that the pressure exerted on the sensor 10f is consistent with that which would be exerted when the subject is standing, but it remains relatively constant, for example 20 seconds, the unit would turn on the delivery of electrical stimulation, so long as the pressure remained relatively constant, as this would be deemed to be a person standing still, with legs and feet not in motion. In this example of a preferred embodiment, it can be readily be appreciated that the motion-pressure sensor 10f could replace the motion sensor or accelerometer 10a, however some preferred embodiments of the invention include both, or in any combination with other motion sensor devices, and the information from them can be integrated by the controller or processor 10b, and compared with an integrated map that contains both acceleration, pressure criteria and perhaps other motion indicia, that determines with greater accuracy the motive condition of the subject's legs and feet and the appropriateness of providing electrical stimulation.

FIG. 5 illustrates a preferred embodiment of the invention where the NMES unit 10 has been located on the top of the foot, where it is less likely to be brushed into something in the course of walking. In this preferred embodiment the NMES unit 10 is detachably attached to the footwear 13 with complementary patches 14a, 14b of hook and loop fastener material, one of course being more permanently attached to the NMES unit 10 and the other to the footwear 13. The footwear can be of any type and can be worn with or without socks or stockings. If worn with stockings, the footwear would have incorporated into them electrodes that could project through the material of the sock or stocking.

The material from which the footwear 13 is made could be partly or completely elastic which would assist in pressing the electrodes against the bottom of the foot. Also this material might be the same or similar to that used for compression stockings for the treatment and prevention of DVT. It is believed that the combination of compression stockings and periodic electrical stimulation of the plantar muscle may have a synergistic effect on the reduction or prevention of DVT. In some applications this footwear would be disposable. For example the footwear 13 might be a disposable slipper given to an airplane passenger with NMES attached. The passenger would take his shoes off, and put the slippers on, adjust the intensity setting to the level that just gently contracts the foot muscles, relax and enjoy the gentle foot message. At the end of the trip the NMES unit could be removed by pulling away the hook and loop detachable attachments 14a and 14b and removing the two connectors 7c. The slipper could then be disposed of and the NMES unit 10 be retained for the next passenger. If however the footwear 13 is worn without a sock or stocking, it is possible that it would function and have those features as that preferred embodiment illustrated in FIG. 3, above. If worn without a sock or stocking, the footwear illustrated in FIG. 5 could have the standard self-adhering disposable electrode either incorporated into the footwear, in which case it would likely be disposable, or be separate, in which case only the electrodes likely would be disposed of after each use. Again the conductors 7a and 7b could be partly or completely incorporated into the material of the footwear 13 or be partly or completely separate.

In the preferred embodiments of the invention illustrated in FIGS. 3, 4 and 5 the NMES unit 10, incorporates either a motion detector 10a, 10f or 10g and controller 10b, memory(s) 10c, timer 10d and perhaps interface unit 10e that processes the motion detector's signals and turns off and on the NMES unit in accordance with a programmed map of sensor signals that connote sufficient or insufficient presumed motion of the calf muscle. While the preferred embodiment of the invention may include all these components, other preferred embodiments of the invention might contain only a subset of them and still be within the ambit of the invention.

While the invention has been described in connection with one item of footwear and reference is made to a single foot, it is to be understood that the method and system that comprise the invention can and in most cases is used on both feet at the same time.

While the invention has been described above in connection with the particular embodiments and examples, one skilled in the art will appreciate that the invention is not necessarily so limited. It will thus be understood that numerous other embodiments, examples, uses, modifications of, and departures from the teachings disclosed may be made, without departing from the scope of the present invention as claimed herein.

For example, it is not necessary that the invention be practiced utilizing the precise pulses per second, ramp up times, stimulus cycles, or stimulus durations, comprising the electrical stimulation routine. These will vary depending upon the user's health and physiological make-up as well, as his special sensitivities. It is therefore apparent that many combinations of electrical stimulus perimeters will achieve successful results so long as the electrodes are placed in a configuration that will stimulate the plantar muscle and that the electrical stimulation routine is modified by the detection and processing of motion in the foot or leg or both.

What is claimed is:

1. A method and a device for the treatment and prevention of circulatory ailments including deep vein thrombosis (DVT) comprising:
placing electrodes on different parts of the intrinsic muscles, on the plantar surface of the foot, or proximate to them, and
applying electrical current with a NMES unit 10, in accordance with a electrical stimulation routine to said electrodes, and
such electrical current causes mild contraction of the foot muscles, thereby increasing blood flow and preventing blood pooling in the calf veins, and
said electrical stimulation routine is altered, supplemented or interrupted, or a combination thereof when motion detection sensors indicate directly or indirectly that the muscles of the calf are in motion,
to optimize the delivery of electrical stimulation to minimize electrical usage, minimize accommodation, perhaps turn off the stimulation during subject ambulation, and maximize blood flow to prevent DVT, all for the particular subject or class of subjects for whom the treatment and device are designed

2. A device for the treatment of prevention of circulatory ailments including deep vein thrombosis (DVT) comprising:
a NMES unit 10, together with electrodes 6a, 6b that deliver electrical stimulation to the foot muscles, thereby increasing blood flow and preventing blood pooling in the calf veins, and
such MMES unit includes controlling means that delivers said electrical stimulation in accordance with an electrical stimulation routine, and
such NMES unit includes or communicates with motion sensing means to detect motion, directly or indirectly, of part or all of the foot and/or calf muscle and such motion sensing means communicates this information to the controller, and
said controller has associated memory element(s) and timing element(s) that adjust, supplement or interrupt, or a combination thereof, the electrical stimulation routine, that may be contained in memory,
by comparing a map of putative inputs that would connote motion of the foot and/or calf muscles, contained in said memory element(s) with actual motion inputs, and
directing the NMES device to deliver or not deliver electrical stimulation to the foot muscles based on the comparison and an algorithm contained in memory, and
such algorithm is programmed to optimize the delivery of electrical stimulation to minimize electrical usage, minimize accommodation, perhaps turn off the stimulation during subject ambulation, and maximize blood flow to prevent DVT, all for the particular subject or class of subjects for whom the treatment and device are designed.

3. The method of claim 1 where the electrical current is applied in cooperation with an electrical stimulation schedule.

4. The method of claim 1, where the motion detection means is comprised of a motions sensor(s) in cooperation with a processor or controller 10b.

5. The method of claim 1, where the processor or controller 10b contains or is in communication with a memory element 10c that contains a map of putative sensor inputs against which the processor or controller 10b can compare actual motion sensor inputs to determine whether the calf muscle is moving sufficiently to prevent DVT, or whether the calf muscle is not moving sufficiently to prevent DVT, and
the said processor and controller 10b uses this information, perhaps in cooperation with an electrical stimulation schedule, to control the application of electrical stimulation, so as to prevent DVT.

6. The method of claim 1, where the motion sensor means is an accelerometer, a strain sensor, a pressure sensor, a GPS unit or some combination thereof.

7. The method of claim 1, where the strain sensor is attached to the foot, ankle, or calf muscle, or combination thereof.

8. The method of claim 1, where the pressure-motion sensor is applied to the bottom of the foot.

9. The method of claim 1, where the motion of the foot, lower leg and calf muscle can be inferred from the pressure-motion sensor signals, the accelerometer sensor, or the strain sensor, or some combination thereof.

10. The method of claim 1, where the processor or controller turns off the application of electrical stimulation during periods of calf muscle activity that is presumed to be sufficient to prevent DVT, without the application of the said electrical stimulation.

11. The method of claim 1, where the application of electrical stimulation is interrupted where it is inferred by sensor inputs that the subject is walking, running or moving in such a manner that the application of muscle stimulation could affect the safety or comfort of the subject.

12. The method of claim 1, where the map can be reprogrammed via an interface 10e.

13. The method of claim 1, where the timer 10d can time the sensor inputs.

14. The method of claim 1, where the application of electrical stimulation can be interrupted to conserve energy and extend battery life.

15. The method of claim 1, where the application of electrical stimulation can be minimized to enhance the user's comfort.

16. The method of claim 1, where the application of electrical stimulation is combined with compressive or graduated compressive stockings.

17. The method of claim 1, where the application of electrical stimulation is combined with a cast or support that immobilizes the lower foot and leg or both.
